(19) United States
(12) Patent Application Publication
(10) Pub. No.: US 2019/0135658 A1
(43) Pub. Date: May 9, 2019

(54) UV IRRADIATION APPARATUS

(71) Applicant: PHOTOSCIENCE JAPAN CORPORATION, Tokyo (JP)

(72) Inventor: Yuji YAMAKOSHI, Taito-ku (JP)

(21) Appl. No.: 16/097,338

(22) PCT Filed: May 16, 2017
(86) PCT No.: PCT/JP2017/018426

§ 371 Date: Oct. 29, 2018

(30) Foreign Application Priority Data
May 31, 2016 (JP) 2016-108587

(51) Int. Cl. C02F 1/32; A61L 2/10; A61L 2/26; B08B 7/00; B08B 3/12

(52) U.S. Cl. CPC C02F 1/325; A61L 2/10; A61L 2/26; B08B 7/0057; B08B 3/12; A61L 2202/11; C02F 2201/3223; A61L 2202/14; A61L 2202/17; C02F 2303/04; C02F 2201/3228; C02F 2201/324; A61L 2202/122

(57) ABSTRACT

The present invention includes a UV-transmissive protective tube accommodated in a pressure-resistant container, a UV lamp accommodated in the protective tube, and a UV-transmissive water passage tube accommodated in the container. Liquid to be treated flows in the water passage tube. The remaining space in the container is filled with a UV-transmissive liquid medium, and UV rays from the UV lamp are radiated to the to-be-treated liquid through the protective tube, the liquid medium, and the water passage tube. The water passage tube is placed in the liquid medium, and thus, pressures inside and outside the water passage tube are substantially equal, in such a manner that the water pressure resistance of the water passage tube is substantially equal to the water pressure resistance of the container, and as a result, there is no need to particularly increase the strength of the water passage tube.

### TECHNICAL FIELD

The present invention relates generally to ultraviolet or UV irradiation apparatus for sterilizing liquid to be treated (to-be-treated liquid) by using UV rays, and more particularly to a UV sterilizer apparatus for use, for example, in water purification plants to deactivate chlorine-resistant disease-causing organisms, such as cryptosporidium, in pure water production plants, and in other water treatment plants.

### BACKGROUND ART

UV irradiation apparatus employed for water treatment include an internal radiation type and an external radiation type. The internal radiation type includes a UV-transmissive protective tube, such as a quartz tube, inserted in a cylindrical stainless steel container, and a UV-emitting lamp accommodated in the protective tube as a light source. The internal radiation type is constructed in such a manner that to-be-treated water is caused to flow in the stainless steel container so that UV rays having passed through the protective tube are radiated to the to-be-treated water within the stainless steel container. Namely, the protective tube for the UV light source is provided in contact with the to-be-treated water within the container. On the other hand, the external radiation type includes a UV light source provided around a UV-transmissive water passage tube (such as a fluorine resin tube or a quartz tube) with a space interposed therebetween. The external radiation type is constructed in such a manner that to-be-treated water is caused to flow through the UV-transmissive water passage tube so that UV rays from the UV light source are radiated to the to-be-treated water within the stainless steel container through the surrounding space and the wall of the water passage tube. Namely, the UV rays are radiated from outside the water passage tube in which the to-be-treated water is caused to pass. Patent Literature 1 below discloses an example of the internal-radiation-type UV irradiation apparatus, and Patent Literature 2 below discloses an example of the external-radiation-type UV irradiation apparatus.

### PRIOR ART LITERATURE

### Patent Literature

Patent Literature 1: Japanese Patent Application Laid-open Publication No. 2007-275825

Patent Literature 2: Japanese Patent Application Laid-open Publication No. 2001-120235

Generally, UV irradiation apparatus presently used in water purification plants are of the internal radiation type. In the case of the internal radiation type, when the protective tube (quartz tube) is damaged or broken, broken pieces of the protective tube and the light source lamp and substances within the light source lamp may flow together with to-be-treated water. Because, in general, a mercury lamp is used as the light source lamp, it has to be assumed that mercury may be mixed into the to-be-treated water, if the protective tube (quartz tube) is damaged or broken. Thus, in the water purification plants, a strainer is inserted at a front stage of the UV irradiation apparatus in order to forestall damage or breakage of the protective tube (quartz tube), particularly to prevent a pebble, which may damage or break the protective tube (quartz tube), from flowing into the UV irradiation apparatus. Further, another strainer is provided at a rear stage of the UV irradiation apparatus in case of damage or breakage of the protective tube (quartz tube). In addition, a tank is provided rearward of the rear strainer of the UV irradiation apparatus in case of a situation where the mercury dissolves into the to-be-treated water, and control is performed to automatically close a valve, provided at the exit of the tank, in response to a water leakage signal generated from the irradiation apparatus.

In the case of the external radiation type, on the other hand, even when the water passage tube of the UV irradiation apparatus is damaged or broken, the only possible glitch is that water spurts out of the water passage tube, and there may not arise a situation where broken pieces of the water passage tube and the mercury within the lamp get mixed into the to-be-treated water. Thus, for the external radiation type, there is no need to provide front and rear strainers, rear tank, automatic valve at the exit of the tank, etc. as mentioned above. However, although water pressure resistance performance required in water treatment plants as well as in water purification plants is 1 MPa, it is difficult for the UV-transmissive water passage tube of the external-radiation-type UV irradiation apparatus to achieve the water pressure resistance performance of 1 MPa, because the UV-transmissive water passage is formed of fluorine resin or quartz. In order to achieve the water pressure resistance performance of 1 MPa with the UV-transmissive water passage tube formed of fluorine resin or quartz, it is necessary to reduce the diameter of the water passage tube and increase the wall thickness of the water passage tube. However, because increasing the wall thickness of the water passage tube lowers the UV transmittance and reducing the diameter of the water passage tube reduces the amount of water that can be treated, the number of the tube to be used for the water treatment has to be increased. Such arrangements require high costs and thus are unrealistic or impractical.

### SUMMARY OF INVENTION

In view of the foregoing problems, it is an object of the present invention to provide a UV irradiation apparatus which can, like an external-radiation-type UV irradiation apparatus, eliminate a need to provide various equipment as measures against possible occurrence of damage or breakage of a protective tube (quartz tube), having a UV lamp inserted therein, while possessing water pressure resistance performance equivalent to that of an internal-radiation-type UV irradiation apparatus.

In order to accomplish the aforementioned object, a UV irradiation apparatus of the present invention includes: a pressure resistant container; a UV-transmissive protective tube accommodated in the container; a UV lamp accommodated in the protective tube; and a UV-transmissive water passage tube accommodated in the container and constructed in such a manner that to-be-treated liquid is caused to flow through the water passage tube. The remaining space within the container is filled with a UV-transmissive liquid medium. UV rays from the UV lamp are radiated to the to-be-treated liquid through the protective tube, the liquid medium, and the water passage tube.

With the UV-transmissive water passage accommodated in the container and constructed in such a manner that the to-be-treated liquid is caused to flow through the water passage tube and with the remaining space within the container being filled with the UV-transmissive liquid medium, pressures inside and outside the water passage tube are kept substantially equal, and a liquid pressure produced in the water passage tube is substantively loaded to the wall of the container located outside the water passage tube. Thus, water pressure resistance performance of the water passage tube substantially equals that of the container, and as a result, it is possible to substantively ensure 1 MPa or over as water pressure resistance performance of a liquid treatment system, by constructing the container to possess sufficient water pressure resistance performance and without particularly strengthening a material, structure, etc. of the water passage tube. Further, because UV-transmissive liquid (such as pure water) is used as the liquid medium that fills the remaining space within the container, all or most of the UV rays radiated from the UV lamp within the protective tube transmit, through the liquid medium, to reach the to-be-treated liquid present in the water passage tube, and as an result, efficient liquid treatment (such as sterilization) can be performed. Furthermore, even when the protective tube having the UV lamp accommodated therein is damaged or broken, broken pieces of the protective tube stay in the liquid medium without reaching the interior of the water passage tube, and therefore, there is no need to provide various equipment as measures against possible occurrence of damage or breakage of the protective tube (quartz tube). In this way, the present invention achieves advantageous benefits of both the internal radiation type and the external radiation type. Note that the UV rays irradiated from the UV lamp may be of any suitable wave length as long as the wave length belongs to a wavelength range necessary for the treatment of the to-be-treated liquid (for example, about 190 nm to 400 nm in the case of sterilization). Also, the liquid medium may be of any suitable UV-transmission performance as long as the UV-transmission performance is sufficient with respect to the wavelength range of the UV rays irradiated from the UV lamp employed.

### BRIEF DESCRIPTION OF DRAWINGS

FIG. 1A is a perspective view of a UV irradiation apparatus according to an embodiment of the present invention, and FIG. 1B is a cross-sectional view of the UV irradiation apparatus;

FIG. 2A is a schematic view showing an example of a structure for supplying to-be-treated liquid to a plurality of water passage tubes within a container, and FIG. 2B is a schematic view showing another example of the structure;

FIG. 3A is a perspective view of a UV irradiation apparatus according to another embodiment of the present invention, and FIG. 3B is a cross-sectional view of the UV irradiation apparatus according to the other embodiment; and

FIG. 4 is a view showing at an enlarged scale an example of a cooling means for cooling a liquid medium.

### DESCRIPTION OF EMBODIMENTS

FIG. 1A is a perspective view of a UV irradiation apparatus according to an embodiment of the present invention, and FIG. 1B is a cross-sectional view (diametric sectional view) of the UV irradiation apparatus. A container 1 is a generally sealed, pressure-resistant stainless-steel container having a cylindrical overall shape, and this container 1 is constructed to withstand a pressure of 1 MPa or over. Note that the container 1 may be of any desired shape without being limited to the cylindrical shape. Further, whereas the cylindrical container 1 is shown in the illustrated example as being disposed in a horizontal orientation, the container 1 may be disposed in a vertical orientation.

A UV-transmissive protective tube 2 is accommodated at a predetermined position (a position along the center axis of the cylindrical protective tube 2 in the illustrated example) within the container 1. Preferably, the protective tube 2 has an elongated cylindrical shape extending in the axial direction of the cylindrical container 1, and the protective tube 2 is detachably attached to the container 1 from the side of one end surface 1a of the cylindrical container 1. A portion of the one end surface 1a of the container 1 to which the protective tube 2 is attached is constructed in a liquid-tight manner such that a liquid medium 5 within the container 1 does not ooze out from the container 1. A UV lamp 3 is detachably accommodated in the protective tube 2 via one end portion 2a of the protective tube 2. As an example, the UV lamp 3 has a linear shape elongated along the length of the protective tube 2. Needless to say, the UV lamp 3 may be of any desired shape other than a linear shape, such as a ring shape or a spherical shape. In any case, the protective tube 2 is shaped to suit the shape of the UV lamp 3. Of the protective tube 2, a portion accommodated in the container 1 is formed of a material, such as quartz glass, that has sufficient UV transmissivity, and a portion (end portion 2a) projecting out of the container 1 is formed of a suitable material (such as metal). Needless to say, the number of the protective tube 2 (and hence the UV lamp 3) accommodated in the container 1 is not limited to just one as in the illustrated example and may be any desired plural number.

Further. UV-transmissive water passage tubes 4 are accommodated in the container 1. In the illustrated example of FIG. 1, four water passage tubes 4, each extending straight in the axial direction of the container 1, are arranged parallel to one another and concentrically around the outer circumference of the protective tube 2 that is the UV-light-source protective tube. The number and shape of the water passage tubes 4 may be any desired number and shape without being limited to those of the illustrated example. Further, the water passage tubes 4 may be formed of fluorine resin, such as an FEP (that is a tetrafluoroethylene-hexafluoropropylene copolymer). Liquid to be treated (to-be-treated liquid) 6 is caused to flow from the outside into the water passage tubes 4.

As illustratively shown in FIG. 1A, each of the water passage tubes 4 extends through the cylindrical container 1 in the axial direction of the container. Of each of the water passage tubes 4, a portion accommodated in the container 1 is formed of a material, such as the above-mentioned fluorine resin, having sufficient UV transmissivity, and opposite end portions 4a and 4b exposed outside the opposite ends of the container 1 are formed of a suitable material (such as metal). Further, in each of the water passage tubes 4, the opposite end portions 4a and 4b may each be equipped with, as necessary, a connection structure for detachably connecting thereto an external tube path (not shown) for directing the to-be-treated liquid 6 into the water passage tube 4. Similarly to the aforementioned, portions of the opposite end surfaces 1a and 1b of the container 1 to which the individual water passage tubes 4 are attached are constructed in a liquid-tight manner such that the liquid medium 5 within the container 1 does not ooze out from the container 1.

As shown in FIG. 1B, the remaining space within the container 1, i.e., the space other than where the protective tube 2 and the water passage tubes 4 are located, is filled with the UV-transmissive liquid medium 5. Pure water, ion-exchanged water, ultrapure water, or the like can be used as the UV-transmissive liquid medium 5. Further, it is preferable that the liquid medium 5 have an UV transmittance of 95% or over (i.e., a UV absorption rate of 5% or less) although the present invention is not so limited. With the remaining space within the container 1 filled with the UV-transmissive liquid medium 5 as noted above, as the to-be-treated liquid 6 is caused to flow through the water passage tubes 4, pressures inside and outside the water passage tubes 4 are kept substantially equal to each other, and a liquid pressure produced in the water passage tubes 4 is substantively loaded to the wall of the container 1 located outside the water passage tubes 4. Thus, water pressure resistance performance of the water passage tubes 4 becomes substantially equal to that of the container 1. As a result, it is substantively possible to substantively ensure 1 MPa or over as water pressure resistance performance of a treatment system by constructing the container 1 to possess sufficient water pressure resistance performance, without particularly strengthening the material, structure, etc. of the water passage tubes 4. Thus, the size, such as the diameter, of the water passage tubes 4 can be easily increased with no particular consideration of the water pressure resistance performance of the water passage tubes 4 themselves. Note that because the protective tube 2 is required to have water pressure resistance performance equivalent to that of the treatment system, the protective tube 2 is constructed to have water pressure resistance performance of 1 MPa or over. Although not shown in the drawings, an inlet/outlet opening may be provided, as necessary, in the container 1 for pouring/discharging the liquid medium 5 into/out of the interior space of the container 1.

In liquid treatment processing by the UV irradiation apparatus arranged in the aforementioned manner, all or most of the UV rays irradiated from the UV lamp 3 transmit through the liquid medium 5 in the container 1 to reach the to-be-treated liquid 6 present within the water passage tubes 4, and as a result, efficient liquid treatment (such as sterilization) can be performed on the to-be-treated liquid 6. Further, even when the protective tube 2 having the UV lamp 3 accommodated therein is damaged or broken, broken pieces of the protective tube 2 stay in the liquid medium 5 without reaching the to-be-treated liquid 6 present within the water passage tubes 4, and therefore, there is no need to provide various equipment as measures against possible occurrence of damage or breakage of the protective tube 2, such as front and rear strainers of the container 1, a rear-stage tank, and an automatic valve at the exist of the tank.

Assuming that the container 1 has an inner diameter of about 210 mm and a length of about 1,000 mm, for example, the protective tube 2 has an outer diameter of about 30 mm, and the water passage tubes 4 each have an outer diameter of 60 mm. In such a case, a low-pressure mercury lamp of about 65 watt can be used as the UV lamp 3.

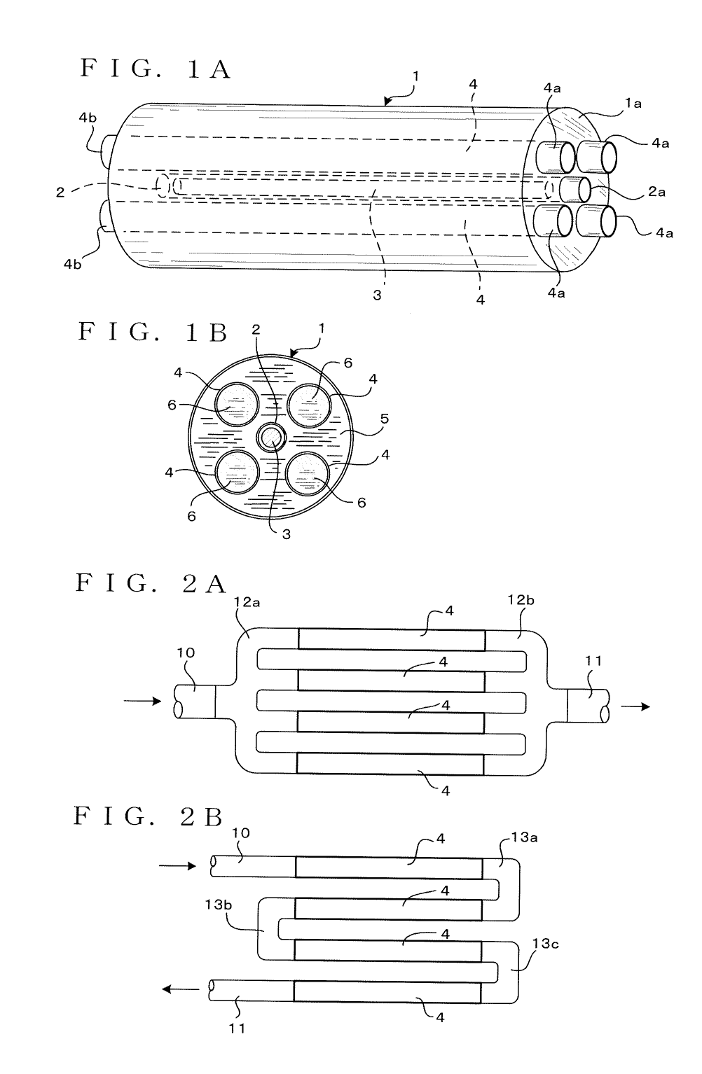

A structure for supplying the to-be-treated liquid 6 to the plurality of water passage tubes 4 accommodated in the container 1 may be constructed as desired from a design point of view. For example, as shown schematically and conceptually in FIG. 2A, an adapter 12a for branching the to-be-treated liquid 6 from a single supply tube path 10, which supplies the to-be-treated liquid 6, to the four water passage tubes 4 may be provided on respective one end portions of the four water passage tubes 4, and another adapter 12b for combining the to-be-treated liquid 6, having got out of the four water passage tubes 4, into a single discharge tube path 11 may be provided at respective other end portions of the four water passage tubes 4. With such arrangements, the UV rays from the UV lamp 3 are radiated in a parallel manner to the to-be-treated liquid 6 flowing through the individual water passage tubes 4. As another example, as shown schematically and conceptually in FIG. 2B, a single supply tube path 10, which supplies the to-be-treated liquid 6, may be connected to one end of one of the water passage tubes 4 (i.e., first water passage tube 4), the other end of the one water passage tube 4 and one end of another one of the water passage tubes 4 (i.e., second water passage tube 4) may be interconnected via an adapter 13a, the other end of the second water passage tube 4 and one end of still another one of the water passage tubes 4 (i.e., third water passage tube 4) may be interconnected via an adapter 13b, the other end of the third water passage tube 4 and one end of still another one of the water passage tubes 4 (i.e., fourth water passage tube 4) may be interconnected via an adapter 13c, and the other end of the fourth water passage tube 4 may be connected to a single discharge tube path 11. Thus, the plurality of water passage tubes 4 are connected together in series. With such arrangements, the UV rays from the UV lamp 3 are repeatedly radiated to the to-be-treated liquid 6 flowing through the serially connected water passage tubes 4. As still another example, the to-be-treated liquid 6 may be supplied separately to the individual water passage tubes 4 via respective supply tube paths 10, and the liquid 6 output from the individual water passage tubes 4 after having been subjected to the treatment may be discharged separately to a plurality of discharge tube paths 11.

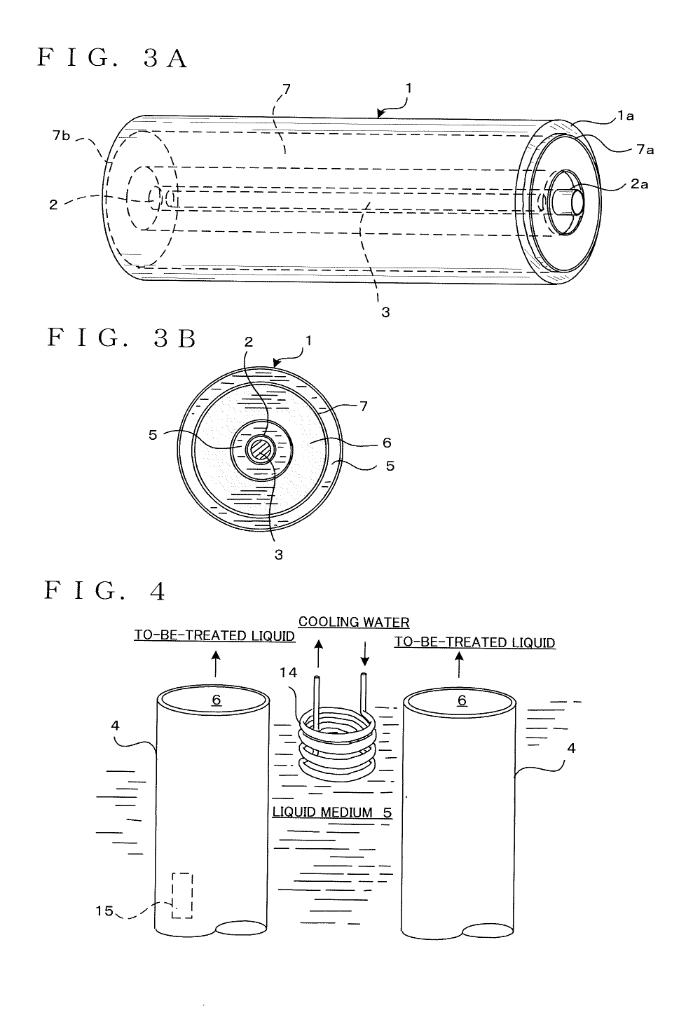

FIG. 3A is a perspective view of a UV irradiation apparatus according to another embodiment of the present invention, and FIG. 3B is a cross-sectional view (diametric sectional view) of the UV irradiation apparatus. As in the illustrated example of FIGS. 1A and 1B, the container 1 is a generally sealed, pressure-resistant, stainless-steel container having a cylindrical shape, and the UV-transmissive protective tube 2 and the UV lamp 3 accommodated in the tube 2 each have a linear shape extending along the central axis line of the container 1. Only one UV-transmissive water passage tube 7 is accommodated in the container 1, and the UV-transmissive water passage tube 7 is constructed as a double tube having a ring cross-sectional shape. More specifically, the to-be-treated liquid 6 is caused to flow through the ring cross-sectional tube path of the water passage tube 7 (i.e., an outer tube path of the double tube), and the light-source protective tube 2 is disposed in an inner space of the water passage tube 7. As in the above-described illustrated example of FIGS. 1A and 1B, the remaining space, i.e. the space other than where the protective tube 2 and the water passage tube 7 are located, is filled with the UV-transmissive liquid medium 5. Namely, in this embodiment, outer and inner spaces of the water passage tube 7 are filled with the UV-transmissive liquid medium 5.

In the embodiment of FIGS. 3A and 3B too, the remaining space within the container 1 is filled with the UV-transmissive liquid medium 5. Thus, as the to-be-treated liquid 6 is caused to flow through the water passage tube 7, pressures inside and outside the water passage tube 7 are kept substantially equal to each other, and a liquid pressure produced within the water passage tube 7 is substantively loaded to the wall of the container 1 located outside the water passage tube 7. Thus, the water pressure resistance performance of the water passage tube 7 becomes substantially equal to that of the container 1. As a result, it is possible to substantively ensure 1 MPa or over as the water pressure resistance performance of the treatment system by merely constructing the container 1 to possess sufficient water pressure resistance performance, without particularly strengthening the material, structure, etc. of the water passage tube 7. Thus, the size of the water passage tube 7 can be easily increased with no particular consideration of the water pressure resistance performance of the water passage tube 7 itself. Furthermore, in the liquid treatment processing performed by the embodiment of FIG. 3, like in the liquid treatment processing performed by the embodiment of FIG. 1, all or most of the UV rays emitted from the UV lamp 3 transmit through the liquid medium 5 within the container 1 to reach the to-be-treated liquid 6 present within the water passage tube 7, and as an result, efficient liquid treatment (such as sterilization) can be performed. Furthermore, even when the protective tube 2 having the UV lamp 3 accommodated therein is damaged or broken, broken pieces of the protective tube 2 stay in the liquid medium 5 without reaching the to-be-treated liquid 6 present within the water passage tube 7, and therefore, there is no need to provide various equipment as measures against possible occurrence of damage or breakage of the protective tube (quartz tube) 2, such as front and rear strainers of the container 1, a rear-stage tank, and an automatic valve at the exist of the tank.

In each of the above-described embodiments, it is preferable that a reflective layer formed for example of aluminum, PTFE (polytetrafluoroethylene) fluorine resin, etc. for effectively reflecting UV rays be provided on the inner wall of the container 1. In this case, UV rays reflected by the reflective layer are radiated to the surface of the water passage tubes 4 or water passage tube 7 located opposed to the light source (UV lamp 3), and as a result, efficient UV irradiation can be performed on the entire to-be-treated liquid 6 passing through the water passage tubes 4 or water passage tube 7.

Next, a description will be given of the performance and capabilities of the UV lamp 3 employed in the above-described embodiments of the present invention. Wavelengths effective for inactivation, by UV rays, of disease-causing organisms and microorganisms are 400 nm and less. In the present invention, UV rays are radiated to the to-be-treated water 6 through the layer of the liquid medium 5, such as pure water, ion-exchanged water, or ultrapure water, which has high UV transmissivity. Because water absorbs UV rays having wavelengths of 190 mm and less, it is not necessary to construct the UV lamp 3 to possess a capability for radiating UV rays having wavelengths of 190 nm and less. Thus, a wavelength range of UV rays that is effective in the present invention is 190 nm to 400 nm, and the UV lamp 3 only has to have a capability for irradiating UV rays that are of any wavelength or wavelength band belonging to the wavelength range of 190 nm to 400 nm. Particularly, because wavelengths of about 200 nm to 300 nm are effective, it is preferable to employ a UV lamp 3 having such a radiation capability. Any desired one of various light sources, such as a mercury lamp like a low-pressure mercury lamp, medium-pressure mercury lamp or high-pressure mercury lamp, a xenon lamp, a flash lamp, and a UV-LED, may be used as a specific example of the light source, although the light source used in the invention is not limited to the above-mentioned light sources.

The following describe materials and shapes of the water passage tubes 4 and 7, as well as positional relationships of the water passage tubes 4 and 7 with the light source. One of various conditions required of each of the water passage tubes 4 and 7 is that UV rays are transmitted through the water passage tube. Examples of a material that satisfies such a condition include a single material, such as quartz, sapphire, or FEP or PFA (tetrafluoroethylene-perfluoro alkyl vinyl ether copolymer) fluorine resin, a compound material, such as quartz or sapphire covered with fluorine resin (where the quartz or sapphire and the fluorine resin may be adhered to each other by thermal shrinkage, or may be absorbed or joined to each other after processing of their respective contact surfaces), and the like. The shape of the water passage tube is not limited to the cylindrical shape as shown in FIG. 1 or the double tube shape (having a ring cross-sectionals shape). For example, the linear water passage tube 4 may have any desired cross-sectional shape other than a circular cross-sectional shape, such as a triangular or rectangular cross-sectional shape. Further, the double-tube-shaped water passage tube 7 may be replaced with a spiral-shaped water passage tube, and the protective tube 2 having the UV lamp 3 accommodated therein may be disposed in a central space of the spiral. Furthermore, the axis of the protective tube 2 having the UV lamp 3 accommodated therein and the axis of the water passage tube 4 or 7 do not have to be parallel to each other and may be perpendicular or oblique to each other. Moreover, whereas FIG. 1 illustrates the example where an inlet/outlet of the water passage tube 4 and an inlet/outlet of the protective tube 2 through which the lamp 2 is inserted into or taken out of the protective tube 2 are located in the same end plane, the present invention is not so limited. In view of enhanced maintenability, however, alternative arrangements may be made such that the inlet/outlet of the water passage tube 4 and the inlet/outlet of the protective tube 2 are not located in the same plane; for example, the alternative arrangements may be such that the lamp 3 can be inserted into or taken out of the protective tube 2 at a curved surface (side surface) of the cylindrical container 1 with the protective tube 2 inclined (obliquely or perpendicularly) relative to the container 1. Such alternative arrangements can facilitate replacement of the lamp 3.

Next, a description will be given of a material, shape, etc. of the protective tube 2. Predetermined UV transmissivity and pressure resistance are required of the protective tube 2 in which the UV lamp 3 is to be inserted. Therefore, it is preferable that the protective tube 2 be formed of a single material such as quartz, sapphire, or FEP or PFA fluorine resin, that has superior UV transmissivity, or a compound material comprising quartz or sapphire covered with fluorine resin, similarly to the aforementioned water passage tubes 4 and 7. Further, because the water pressure resistance of 1 MPa or over is required of the protective tube 2, it is desirable that the protective tube 2 be formed in a cylindrical shape. In general, tubes formed of fluorine resin are limited in their inner diameter and wall thickness. Thus, in the case where the water pressure resistance of 1 MPa or over is required of the protective tube 2 formed of fluorine resin, it is necessary that the protective tube 2 have a wall thickness of 2 mm or over for an inner diameter of 20 mm, although a necessary wall thickness of the protective tube 2 depends on a temperature. In the case where the protective tube 2 is formed of quartz or sapphire, or a compound material comprising quartz or sapphire covered with fluorine resin, on the other hand, the water pressure resistance of 1 MPa or over can be ensured even with the protective tube 2 having a wall thickness of 1 mm for the inner diameter of 20 mm.

Next, cooling of the liquid medium 5 will be described. The ion-exchanged water used as the liquid medium 5 rises in temperature with heat generated from the low-pressure mercury lamp and reaches a certain water temperature (for example about 60° C. that may, however, differ depending on an ambient temperature, temperature of the to-be-treated liquid 6, and presence/absence of flows of the to-be-treated liquid 6). Further, UV output of the low-pressure mercury lamp varies with an ambient temperature of the lamp and decreases at about 60° C. by about 50 to 70% relative to the UV output at an optimum temperature (ambient water temperature is 25° C.). In order to reduce adverse influences of such an water temperature rise, it is preferable to provide a cooling means within the container 1 to cool the liquid medium 5 by use of the cooling means in such a manner that the protective tube 2 and the UV lamp 3 accommodated in the protective tube 2 can be cooled, thereby preventing the decrease of the UV output.

An example of such a cooling means is shown in FIG. 4 at an enlarged scale. More specifically, FIG. 4 shows an example where the cooling means is applied to an embodiment which has a plurality of linear water passage tubes 4 accommodated in the cylindrical container 1 as in the embodiment of FIG. 1, and in which the cylindrical container 1 is installed in a vertical orientation. The cooling means includes a plurality of spiral pipes 14 disposed in a suitable upper portion of a liquid medium storage space, which stores the liquid medium 5, within the vertically oriented container 1, and cooling water is caused to flow through the spiral pipes 14. Liquid medium cooling efficiency can be enhanced by the cooling spiral pipes 14 being disposed in the upper portion of the liquid medium storage space within the container 1. It was confirmed that, even when one low-pressure mercury lamp of 65 watt is kept illuminated, the temperature of the ion-exchanged water used as the liquid medium 5 can be maintained at about 25° C. by providing, in an upper portion of the container 1, one spiral pipe 14 having an inner diameter of 6 mm, an outer diameter of 8 mm, and a total length of about 4 m, and causing cooling water of a temperature of 20° C. through such a pipe 14 at a flow rate of one liter per minute. With such arrangements, an optimal temperature for maximizing the UV output was obtained. Note that the to-be-treated liquid 6 before being irradiated with UV rays may be used as the above-mentioned cooling water to be caused to flow at the flow rate of one liter per minute. Alternatively, dedicated cooling water may be circulated.

Today, for UV irradiation apparatus employed in water purification plants, it is required by law to constantly measure a UV radiation intensity. For this purpose, it just suffices that a UV sensor 15 be provided within the water passage tube 4 that is a flow path for the to-be-treated liquid 6, as shown in FIG. 4.

Next, a description will be given of influences of the UV reflecting layer provided on the inner wall of the container 1. The to-be-treated liquid 6 within the water passage tube 4 is irradiated with UV rays emitted from the UV lamp 3, after which the UV rays reach the inner wall of the stainless steel container 1. Because the UV reflectance of the stainless steel is approximately 30%, the reflection effect is small if no particular reflective layer is provided. However, with the embodiment of the present invention, where the reflective layer for efficiently using the UV rays having reached the inner wall of the stainless steel container 1 is provided on the inner wall of the container 1, the UV radiation intensity within the water passage tube 4 can be increased. Because the reflective layer is kept in constant contact with pure water, it is desirable that the reflective layer be formed of a material that is not corroded with the pure water. Because the preferred wavelength range in the embodiment is approximately 200 nm to 300 nm, it is preferable that the reflective layer be formed of a material having a high reflectance with respect to the above-mentioned wavelength range. For example, aluminum coated with fluorine resin, or PTFE, FEP or PFA fluorine resin is suitable for reflecting UV rays. As well known, reflection includes specular (or regular) reflection and diffuse (or irregular) reflection, of which the specular reflection occurs in a state where the reflective surface is like a mirror surface while the diffuse reflection occurs in a state where the reflective surface is an uneven surface. The reflective layer may be formed so as to present any one of these reflection effects. As an example, a cylinder formed by winding an FEP sheet having a thickness of 1 mm was closely attached to the inner wall of the container 1, and then, an intensity of UV radiation to the to-be-treated water 6 within the water passage tube 4 was measured. As a result, an increase of the UV radiation intensity that is up to four times as high as a UV radiation intensity in a case where such an FET sheet was not provided could be obtained. The cylinder having such an FEP sheet layer has a circumference of about 660 mm so as to be closely attached to the inner circumference of the stainless steel container 1 having a diameter of 210 mm. In order to obtain such an FEP sheet layer having the circumference of about 660 mm, it is only necessary that an FEP sheet of a rectangular shape having a short side of 700 mm and a long side of 1 m equal to the axial length of the stainless steel container 1 be wound in a short-side direction, and the remaining portion of 40 mm may be left in a natural overlapping state without its overlapping regions having to be welded together. Even in a case where the reflective layer is formed of aluminum, the overlapping regions do not have to be welded together.

Further, the UV reflective surface provided on the inner wall of the container 1 does not have to be fixed to the container 1. Besides, because the liquid medium 5 is present both inside and outside the reflective layer, high water pressure resistance is not required. Thus, the shape of the reflective layer may be chosen with a considerable degree of freedom; for example, the reflective layer may be of a plate shape having flat surfaces rather than a cylindrical shape. Particularly, in a case where a reflective plate constructed to produce specular reflection is used and the reflective plate may be shaped to have a generally elliptical curved surface, and where a light source is provided at one of two focal points, light specularly reflected by the reflective plate focuses at the other focal point. Thus, the light source (protective tube 2), the water passage tube 4, and the reflective plate may be disposed in a positional arrangement utilizing this principle.

Next, a description will be given of anti-freezing and anti-condensation measures. If the UV lamp 3 (such as a low-pressure mercury lamp) is constantly kept on or illuminated, the liquid medium 5 is warmed by the lamp, and thus, there is no possibility of the liquid medium 5 and the to-be-treated liquid 6 being frozen. However, when the lamp 3 is turned off, dew condensation may occur within the protective tube 2 having the lamp 3 inserted therein, and consequently, there may occur problems of the lamp 3 going out and/or a socket connecting the lamp 3 corroding due to the condensation water. Constantly keeping the lamp 3 can also function as anti-condensation measures. However, if the lamp 3 is kept illuminated with the cooling water supply to the spiral pipe 4 stopped during a non-liquid-treatment-processing time, the liquid medium 5 is heated, for example, to approximately 60° C., and thus, the to-be-treated liquid 6, convectively flowing in the water passage tube 4, is also heated. In order to avoid the to-be-treated liquid 6 from being heated due to the constant illumination of the lamp 3 during the non-liquid-treatment-processing time, it suffices that the water passage tube 4 be kept empty during the non-liquid-treatment-processing time.

Cleaning of the water passage tubes 4 and 7 will be described next. It is assumable that dirt and the like adhere to the inner wall surfaces of the water passage tubes 4 and 7. In a case where the UV irradiation apparatus is used in a water purification plant, dirt and the like are more likely to adhere than in a case where the UV irradiation apparatus is used in pure water production. Water having been subjected to membrane treatment used as measures against cryptosporidium in a water purification plant includes permeated water that becomes purified water, and wastewater to be collected or discharged. The wastewater is more likely to cause adherence of dirt and the like than the purified water. It was confirmed that, after ten-year's use of a fluorine resin tube through which the waste water was caused to flow, there was almost no adherence of dirt and the like owning to the effect of non-wettability of the fluorine resin. Thus, using such a fluorine resin tube as the water passage tube 4 (or 7) can be expected to be highly effective in prevention of adherence of dirt and the like.

Further, a cleaning mechanism may be provided for cleaning the interior of the water passage tube 4 (or 7). Such a cleaning mechanism may be based, for example, on at least one of applying ultrasonic waves to the liquid medium 5, imparting mechanical vibrations to the water passage tube 4 (or 7) by use of a vibrator, and cleaning the interior of the water passage tube 4 (or 7) by use of a brush.

1. A UV irradiation apparatus comprising:
a pressure resistant container;
a UV-transmissive protective tube accommodated in the container;
a UV lamp accommodated in the protective tube; and
a UV-transmissive water passage tube accommodated in the container and constructed in such a manner that to-be-treated liquid is caused to flow through the water passage tube,
a remaining space within the container being filled with a UV-transmissive liquid medium,
UV rays from the UV lamp being radiated to the to-be-treated liquid through the protective tube, the liquid medium, and the water passage tube.

2. The UV irradiation apparatus as claimed in claim 1, wherein the liquid medium is pure water.

3. The UV irradiation apparatus as claimed in claim 1, wherein a reflective layer for reflecting the UV rays is provided on an inner wall of the container.

4. The UV irradiation apparatus as claimed in claim 3, wherein the reflective layer is formed of a material using at least one of aluminum and fluorine resin.

5. The UV irradiation apparatus as claimed in claim 1, wherein the UV lamp produces UV rays belonging to a wavelength range of 190 to 400 nm.

6. The UV irradiation apparatus as claimed in claim 1, wherein a UV sensor is provided within the water passage tube.

7. The UV irradiation apparatus as claimed in claim 1, which further comprises a cooling means for cooling the liquid medium within the container.

8. The UV irradiation apparatus as claimed in claim 1, which further comprises a cleaning mechanism for cleaning the water passage tube.

9. The UV irradiation apparatus as claimed in 8, wherein the cleaning mechanism is based on at least one of applying ultrasonic waves to the liquid medium, imparting vibrations of a vibrator to the water passage tube by use of a vibrator, and cleaning an interior of the water passage tube by use of a brush.

10. The UV irradiation apparatus as claimed in claim 2, wherein a reflective layer for reflecting the UV rays is provided on an inner wall of the container.

11. The UV irradiation apparatus as claimed in claim 10, wherein the reflective layer is formed of a material using at least one of aluminum and fluorine resin.

12. The UV irradiation apparatus as claimed in claim 10, wherein a UV sensor is provided within the water passage tube.

13. The UV irradiation apparatus as claimed in claim 10, which further comprises a cooling means for cooling the liquid medium within the container.

14. The UV irradiation apparatus as claimed in claim 3, wherein a UV sensor is provided within the water passage tube.

15. The UV irradiation apparatus as claimed in claim 3, which further comprises a cooling means for cooling the liquid medium within the container.
